(19) United States
(12) Patent Application Publication
(10) Pub. No.: US 2023/0290469 A1
(43) Pub. Date: Sep. 14, 2023

(54) RECORDING MEDIUM, INFORMATION PROCESSING APPARATUS, HEALTHCARE WORKER TERMINAL, INFORMATION PROCESSING SYSTEM AND HEALTHCARE WORKER SYSTEM

(71) Applicant: Konica Minolta, Inc., Tokyo (JP)

(72) Inventors: Moeko Hagiwara, Chikawa-shi (JP); Akira Shimauchi, Tokyo (JP)

(73) Assignee: Konica Minolta, Inc., Tokyo (JP)

(21) Appl. No.: 18/177,759

(22) Filed: Mar. 3, 2023

(30) Foreign Application Priority Data
Mar. 14, 2022 (JP) 2022-039358

(51) Int. Cl. G16H 20/10; G16H 80/00

(52) U.S. Cl. CPC G16H 20/10; G16H 80/00

(57) ABSTRACT

A non-transitory computer readable recording medium storing instructions to control a computer of an information processing apparatus that processes information obtained from a patient terminal connectable to a healthcare worker terminal and to an electronic prescription server that stores electronic prescription information and dispensing information, the instructions causing the computer to execute: obtaining, from the patient terminal, the dispensing information associated with the electronic prescription information; obtaining, from the patient terminal, medicine taking information on a patient; associating the dispensing information and the medicine taking information with each other and creating integrated information; and outputting the integrated information to the healthcare worker terminal or the patient terminal.

### CROSS-REFERENCE TO RELATED APPLICATIONS

The entire disclosure of Japanese Patent Application No. 2022-039358 filed on Mar. 14, 2022 is incorporated herein by reference.

### BACKGROUND

### Technical Field

The present invention relates to a recording medium, an information processing apparatus, a healthcare worker terminal, an information processing system and a healthcare worker system.

### Description of Related Art

Conventionally, in a medical facility such as a hospital or a clinic, prescription information created by a doctor in the facility is accumulated and stored in an electronic health record. The doctor views the prescription information, asks a patient about medicine taking situations and their physical condition, refers to a test result, performs a medical examination, and issues a prescription.

However, past prescription information in the own facility is insufficient for information during a diagnosis.

Since the prescription information created in the own facility and actual dispensing information (information on actual dispensing in a dispensing pharmacy or the like) are not the same, an appropriate diagnosis requires grasping past dispensing information. This is because the prescription information is subjected to change to generics, change due to prescription question by a pharmacist, change in form by a pharmacist and the like.

To improve the accuracy of a medical examination, medicine taking information (information on medicine actually taken by a patient) is also required to be grasped.

In this relation, JP 2013-145567A discloses a system that stores medicine information (dispensing information) and medicine taking information on patients in a server installed in a pharmacy or the like.

A more correct diagnosis further requires grasping dispensing information associated with a prescription issued in a medical facility other than the own facility.

However, with the technology disclosed in JP 2013-145567A, the dispensing information is stored only in the server installed in the pharmacy or the like having provided the patient with medicine. Consequently, the dispensing information and the medicine taking information are not necessarily correctly managed.

### SUMMARY

One or more embodiments of the present invention provide a technological improvement over such conventional technologies as discussed above. In particular, one or more embodiments of the present invention provide a recording medium, an information processing apparatus, a healthcare worker terminal, an information processing system and a healthcare worker system that can more correctly manage the dispensing information and the medicine taking information. This provides a practical, technological improvement over conventional technologies that would be readily appreciated by those skilled in the art, as will be discussed in further detail later.

According to an aspect of the present invention, a recording medium reflecting one aspect of the present invention is:
a non-transitory computer readable recording medium storing instructions to control a computer of an information processing apparatus that processes information obtained from a patient terminal connectable to an electronic prescription server that stores electronic prescription information and dispensing information and to a healthcare worker terminal, the instructions causing the computer to execute:
obtaining the dispensing information associated with the electronic prescription information, from the patient terminal;
obtaining medicine taking information on a patient from the patient terminal;
associating the obtained dispensing information and medicine taking information with each other and creating integrated information; and
outputting the integrated information to the healthcare worker terminal or the patient terminal.

According to an aspect of the present invention, an information processing apparatus reflecting one aspect of the present invention is:
an information processing apparatus processing information obtained from a patient terminal connectable to an electronic prescription server that stores electronic prescription information and dispensing information and to a healthcare worker terminal, the information processing apparatus including a hardware processor that:
obtains the dispensing information associated with the electronic prescription information, from the patient terminal;
obtains medicine taking information on a patient from the patient terminal;
associates the obtained dispensing information and medicine taking information with each other and creates integrated information; and
outputs the integrated information to the healthcare worker terminal or the patient terminal.

According to an aspect of the present invention, a recording medium reflecting one aspect of the present invention is:
a non-transitory computer readable recording medium storing instructions to control a computer of an information processing apparatus that is connected to an electronic prescription server that stores electronic prescription information and dispensing information, to a patient terminal and to a healthcare worker terminal, and processes obtained information, the instructions causing the computer to execute:
obtaining the dispensing information associated with the electronic prescription information, from the electronic prescription server;
obtaining medicine taking information on a patient from the patient terminal;
associating the obtained dispensing information and medicine taking information with each other and creating integrated information; and
outputting the integrated information to the healthcare worker terminal or the patient terminal.

According to an aspect of the present invention is an information processing apparatus reflecting one aspect of the present invention is:
an information processing apparatus that is connected to an electronic prescription server that stores electronic prescription information and dispensing information, to a patient terminal and to a healthcare worker terminal, and processes obtained information, the information processing apparatus including a hardware processor that:
obtains the dispensing information associated with the electronic prescription information, from the electronic prescription server;
obtains medicine taking information on a patient from the patient terminal;
associates the obtained dispensing information and medicine taking information with each other and creates integrated information; and
outputs the integrated information to the healthcare worker terminal or the patient terminal.

According to an aspect of the present invention, a recording medium reflecting one aspect of the present invention is:
a non-transitory computer readable recording medium storing instructions to control a computer of a healthcare worker terminal that processes information obtained from an electronic prescription server that stores electronic prescription information and dispensing information and from a patient terminal, the instructions causing the computer to execute:
obtaining the dispensing information associated with the electronic prescription information, from the electronic prescription server;
obtaining medicine taking information on a patient from the patient terminal;
associating the obtained dispensing information and medicine taking information with each other and creating integrated information; and
outputting the integrated information to a display of the healthcare worker terminal or the patient terminal.

According to an aspect of the present invention, a healthcare worker terminal reflecting one aspect of the present invention is:
a healthcare worker terminal processing information obtained from an electronic prescription server that stores electronic prescription information and dispensing information and from a patient terminal, the healthcare worker terminal including a hardware processor that:
obtains the dispensing information associated with the electronic prescription information, from the electronic prescription server;
obtains medicine taking information on a patient from the patient terminal;
associates the obtained dispensing information and medicine taking information with each other and creates integrated information; and
outputs the integrated information to a display of the healthcare worker terminal or the patient terminal.

According to an aspect of the present invention, an information processing system reflecting one aspect of the present invention is: an information processing system that includes an information processing apparatus processing information obtained from a patient terminal connectable to an electronic prescription server that stores electronic prescription information and dispensing information and to a healthcare worker terminal, the information processing system including a hardware processor that:
obtains the dispensing information associated with the electronic prescription information, from the patient terminal;
obtains medicine taking information on a patient from the patient terminal;
associates the obtained dispensing information and medicine taking information with each other and creates integrated information; and
outputs the integrated information to the healthcare worker terminal or the patient terminal.

According to an aspect of the present invention, an information processing system reflecting one aspect of the present invention is: an information processing system including an information processing apparatus that is connected to an electronic prescription server that stores electronic prescription information and dispensing information, to a patient terminal and to a healthcare worker terminal, and processes obtained information, the information processing system including a hardware processor that:
obtains the dispensing information associated with the electronic prescription information, from the electronic prescription server;
obtains medicine taking information on a patient from the patient terminal;
associates the obtained dispensing information and medicine taking information with each other and creates integrated information; and
outputs the integrated information to the healthcare worker terminal or the patient terminal.

According to an aspect of the present invention, a healthcare worker system reflecting one aspect of the present invention is: a healthcare worker system that includes a healthcare worker terminal processing information obtained from an electronic prescription server that stores electronic prescription information and dispensing information and from a patient terminal, the healthcare worker system including a hardware processor that:
obtains the dispensing information associated with the electronic prescription information, from the electronic prescription server;
obtains medicine taking information on a patient from the patient terminal;
associates the obtained dispensing information and medicine taking information with each other and creates integrated information; and
outputs the integrated information to the healthcare worker terminal or the patient terminal.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a system configuration diagram of an information processing system according to a first embodiment;

FIG. 2 is a block diagram showing a functional configuration of a patient information management server;

FIG. 3 is a block diagram showing a functional configuration of a patient terminal;

FIG. 4 is a block diagram showing a functional configuration of a healthcare worker terminal;

FIG. 5 is a block diagram showing a functional configuration of a pharmacy terminal;

FIG. 6 is a block diagram showing a functional configuration of an electronic prescription server;

FIG. 7 is a flowchart showing information processing;

FIG. 8 shows an example of a medicine taking recording screen displayed on the patient terminal;

FIG. 9 shows an example of a vital recording screen displayed on the patient terminal;

FIG. 10 shows an example of integrated information display screen displayed on the healthcare worker terminal;

FIG. 11 is a system configuration diagram of an information processing system according to a second embodiment; and

FIG. 12 is a system configuration diagram of an information processing system according to a third embodiment.

### DETAILED DESCRIPTION

Hereinafter, referring to the drawings, embodiments of a recording medium, an information processing apparatus, a healthcare worker terminal, an information processing system and a healthcare worker system are described. However, a scope of the invention is not limited to the illustrated examples.

### First Embodiment

### [Configuration of Information Processing System]

First, an information processing system 100 according to a first embodiment is described.

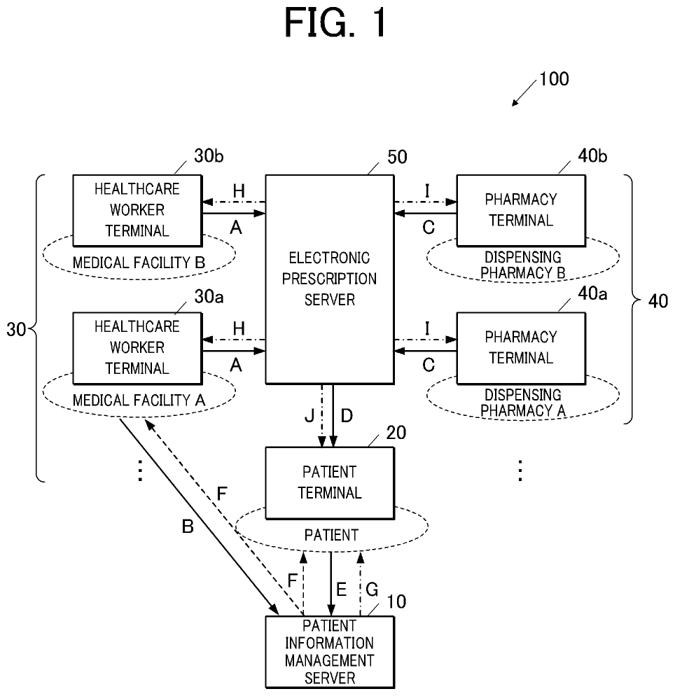

FIG. 1 shows the system configuration of the information processing system 100.

As shown in FIG. 1, the information processing system 100 includes: a patient information management server 10; a patient terminal 20 used by a patient; a healthcare worker terminal 30 used by a medical facility; a pharmacy terminal 40 used by a dispensing pharmacy; and an electronic prescription server 50.

Note that the numbers of patient terminals 20, healthcare worker terminals 30 and pharmacy terminals 40 are not limited.

The electronic prescription server 50 is communicably connected to the patient terminal 20, the healthcare worker terminal 30 and the pharmacy terminal 40 such that data can be communicated via a communication network such as the Internet.

The patient information management server 10 is communicably connected to the patient terminal 20 and the healthcare worker terminal 30 such that data can be communicated via the communication network such as the Internet.

The patient terminal 20 is connectable to the electronic prescription server 50 and the healthcare worker terminal 30 via the communication network such as the Internet.

The patient information management server 10 is an information processing apparatus that accumulates, manages and processes information about health care for patients registered from the patient terminal 20.

The patient information management server 10 is a cloud server provided in a cloud environment, or a local server. For example, the patient information management server 10 is a PHR (Personal Health Record) server.

The patient terminal 20 is a terminal apparatus, such as a smartphone, used by a patient.

A patient application program (hereinafter called a patient application) is installed in the patient terminal 20. The patient performs data communication with the patient information management server 10 and the electronic prescription server 50 via the patient application.

The healthcare worker terminal 30 is a computer apparatus, such as a PC (Personal Computer) or a tablet terminal, used by a healthcare worker (a doctor, a nurse or the like) belonging to a medical facility.

The healthcare worker terminal 30 includes a medical image management apparatus (PACS (Picture Archiving and Communication System)), an electronic health record system and a receipt computer. The medical image management apparatus (PACS) may be an apparatus that integrally includes a radiographing control device (console) and a medical image management device.

A healthcare worker application program (hereinafter called a healthcare worker application) is installed in the healthcare worker terminal 30. The healthcare worker belonging to the medical facility performs data communication with the patient information management server 10 and the electronic prescription server 50 via the healthcare worker application.

The pharmacy terminal 40 is a computer apparatus, such as a PC or a tablet terminal, used by a pharmacist or the like belonging to a dispensing pharmacy.

A pharmacy application program (hereinafter called a pharmacy application) is installed in the pharmacy terminal 40. The pharmacist or the like belonging to the dispensing pharmacy performs data communication with the electronic prescription server 50 via the pharmacy application.

The electronic prescription server 50 accumulates and manages the information about health care for patients registered from the healthcare worker terminal 30 and the pharmacy terminal 40.

The electronic prescription server 50 is a cloud server provided in a cloud environment.

Next, operations of the information processing system 100 shown in FIG. 1 are described.

A doctor (healthcare worker) registers data, such as electronic prescription information, in the electronic prescription server 50 via the healthcare worker application, using the healthcare worker terminal 30 (operation A).

The doctor (healthcare worker) registers data, such as test information, in the patient information management server 10 via the healthcare worker application, using the healthcare worker terminal 30 (operation B).

The pharmacist registers data, such as dispensing information, in the electronic prescription server 50 via the pharmacy application, using the pharmacy terminal 40 (operation C).

The electronic prescription server 50 transmits the dispensing information on the patient to the patient terminal 20 (operation D).

The patient registers data, such as vital information, medicine taking information, and dispensing information received from the electronic prescription server 50, in the patient information management server 10 via the patient application, using the patient terminal 20 (operation E).

The patient information management server 10 transmits (outputs) integrated information that associates the dispensing information and the medicine taking information received from the patient terminal 20 with each other, and statistical information generated based on the integrated information on multiple patients, to the patient terminal 20 and the healthcare worker terminal 30 (operation F).

The patient information management server 10 outputs an alarm for the patient to the patient terminal 20 via the patient application (operation G).

The electronic prescription server 50 permits the healthcare worker terminal 30 to view the information on the patient under medical examination and treatment in the medical facility, via the healthcare worker application (operation H).

The electronic prescription server 50 permits the pharmacy terminal 40 to view information on the patient visiting the dispensing pharmacy via the pharmacy application (operation I).

The electronic prescription server 50 permits the patient terminal 20 to view information on the patient via the patient application (operation J).

### [Configuration of Patient Information Management Server]

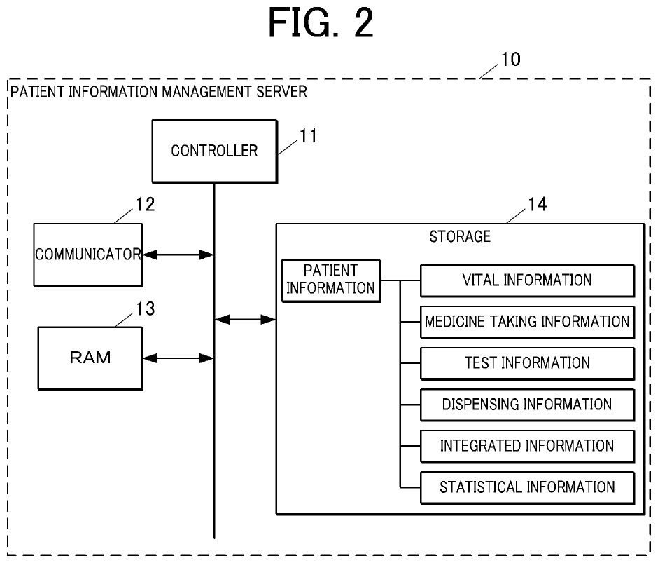

FIG. 2 shows the functional configuration of the patient information management server 10.

As shown in FIG. 2, the patient information management server 10 includes a controller 11 (hardware processor), a communicator 12, a RAM 13 and a storage 14, which are connected to each other via a bus.

The controller 11 includes a CPU (Central Processing Unit), and comprehensively controls processing operations of the components of the patient information management server 10. Specifically, the CPU reads various processing programs (or instructions) stored in the storage 14, loads the processing programs into the RAM 13 and executes various processes in cooperation with the programs.

The communicator 12 includes a network interface, and transmits and receives data to and from an external apparatus connected via the communication network.

The RAM 13 forms a work area that temporarily stores various programs (or instructions) read from the storage 14, input or output data, parameters and the like, during various processes subjected to execution control by the controller 11.

The storage 14 includes an HDD (Hard Disk Drive) or a nonvolatile semiconductor memory, and stores various processing programs (or instructions), and parameters, files and the like required to execute the programs. For example, the storage 14 stores: a web server program for achieving a function as that of a web server that communicates with a web browser installed in an external apparatus through the HTTP protocol and provides the web browser with various web screens; and an application program that operates on the web server and provides a patient information sharing service for an operator of the external apparatus via a web browser.

The storage 14 stores patient information.

The patient information is information on each patient. The patient information includes a patient ID, the name of a patient, the age, the height, the weight, allergic information, the side effect history, the name of an injury or a disease, the names of a regularly visiting medical facility and dispensing pharmacy.

Furthermore, the storage 14 stores vital information, medicine taking information, test information, dispensing information, integrated information, statistical information and the like on the patient in association with the patient information.

The vital information and the medicine taking information are data input by the patient through the patient terminal 20.

The test information is data input by a healthcare worker belonging to the medical facility through the healthcare worker terminal 30.

The dispensing information is data input by a pharmacist belonging to the dispensing pharmacy through the pharmacy terminal 40.

The vital information may be obtained through the patient terminal 20, or obtained through a vital measurement instrument that measures vital information.

The vital information includes the blood glucose level, blood pressure, heart rate, body temperature, and weight.

The medicine taking information is obtained through the patient terminal 20.

The medicine taking information is information on medicine actually taken (ingested) by the patient. The medicine taking information includes information on presence or absence of medicine taking, medicines taken (ingested) by the patient, the medicine taking date and time, and the interval and quantity of medicine taking.

The test information is information held by a specimen testing system, an image management system, an electronic health record system and the like in each medical facility.

The test information is information on tests conducted for the patient (test items, test results, etc.).

The dispensing information is information held by each dispensing pharmacy.

The dispensing information is information about medicines dispensed in conformity with a prescription by a doctor in each dispensing pharmacy (medicines, timing, quantities, etc.).

The integrated information is information that associates the dispensing information with the medicine taking information. The information further includes what is associated with the vital information and/or the test information.

The statistical information is generated by applying statistical processing to integrated information on multiple patients. The statistical processing is, for example, processes that calculate the means, medians, modes and the like in the integrated information on the patients. The processing is processes of calculating values narrowed down with respect to the attributes (gender, age, severity, etc.) of the patients.

The controller 11 obtains the dispensing information associated with the electronic prescription information, from the patient terminal 20. Here, the controller 11 functions as a first obtainer.

The controller 11 obtains medicine taking information on a patient from the patient terminal 20. Here, the controller 11 functions as a second obtainer.

The controller 11 associates the obtained dispensing information and medicine taking information with each other and creates integrated information. Here, the controller 11 functions as an association provider.

The controller 11 outputs the integrated information to the healthcare worker terminal 30 or the patient terminal 20. Here, the controller 11 functions as a first outputter.

The controller 11 obtains vital information from the patient terminal 20. Here, the controller 11 functions as a third obtainer.

The controller 11 generates statistical information, based on the integrated information. Here, the controller 11 functions as a generator.

The controller 11 outputs the statistical information. Here, the controller 11 functions as a second outputter.

### [Configuration of Patient Terminal]

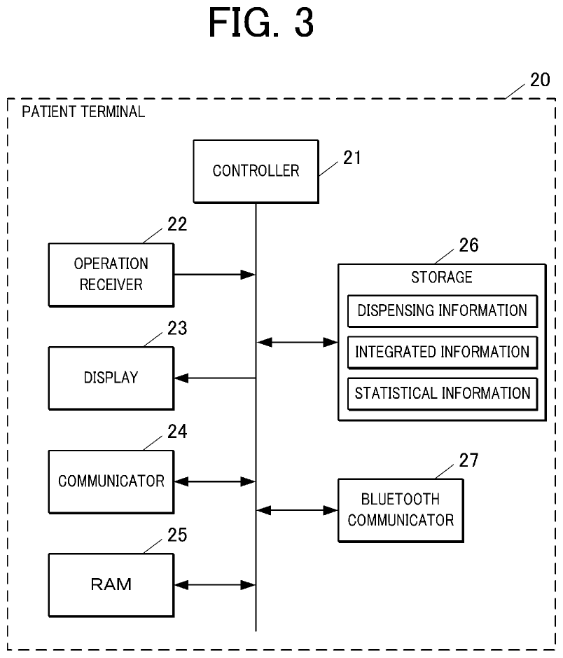

FIG. 3 shows the functional configuration of the patient terminal 20.

As illustrated in FIG. 3, the patient terminal 20 includes a controller 21, an operation receiver 22, a display 23, a communicator 24, a RAM 25, a storage 26 and a Bluetooth communicator 27, which are connected to each other by a bus.

The controller 21 includes a CPU, and comprehensively controls processing operations of the components of the patient terminal 20. Specifically, according to an operation signal input from the operation receiver 22 or an instruction signal received by the communicator 24, the CPU reads various processing programs (or instructions) stored in the storage 26, loads the programs into the RAM 25, and executes various processes in cooperation with the programs.

The operation receiver 22 includes an operation key, such as a power key that turns on and off the power source, and a touch panel stacked on the display 23, and outputs, to the controller 21, an operation signal corresponding to each operation key and an operation signal in accordance with the position of a touch operation of a finger or the like of an operator.

The display 23, which includes a liquid crystal display (LCD), displays various screens in accordance with an instruction of a display signal input from the controller 21. For example, the display 23 displays various web screens, based on data that is received from the patient information management server 10 and is to be displayed on various web screens.

The communicator 24 includes a network interface, and transmits and receives data to and from an external apparatus connected via the communication network. The communicator 24 performs wireless communication using a mobile-phone line or the like, and transmits and receives data to and from an external apparatus connected via a communication network.

The RAM 25 forms a work area that temporarily stores various programs (or instructions) read from the storage 26, input or output data, parameters and the like, during various processes subjected to execution control by the controller 21.

The storage 26 includes an HDD or a nonvolatile semiconductor memory, and stores various processing programs (or instructions), and parameters, files and the like required to execute the programs. For example, the storage 26 stores a web browser program for achieving a web browser, a patient application and the like.

The storage 26 stores the dispensing information received from the electronic prescription server 50.

The storage 26 stores integrated information and statistical information received from the patient information management server 10.

The Bluetooth communicator 27 performs data communication, in conformity with Bluetooth communication scheme, with an external apparatus via an antenna for transmission and reception of Bluetooth communication. For example, the Bluetooth communicator 27 obtains vital information from the vital measurement instrument.

### [Configuration of Healthcare Worker Terminal]

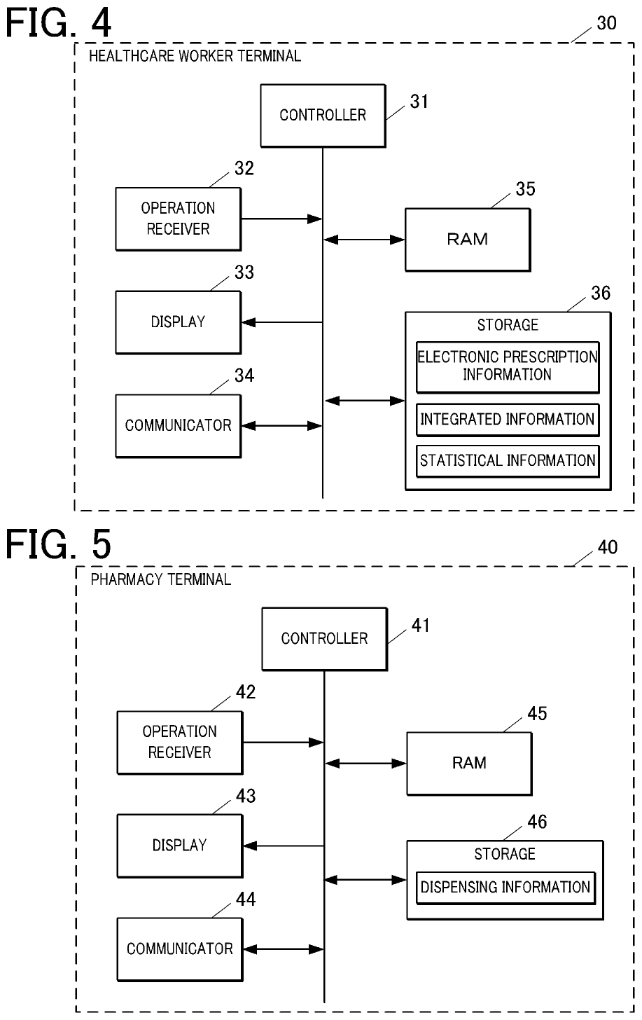

FIG. 4 shows the functional configuration of the healthcare worker terminal 30.

As illustrated in FIG. 4, the healthcare worker terminal 30 includes a controller 31, an operation receiver 32, a display 33, a communicator 34, a RAM 35 and a storage 36, which are connected to each other by a bus.

The controller 31, the display 33, the communicator 34 and the RAM 35 of the healthcare worker terminal 30 are respectively similar to the controller 21, the display 23, the communicator 24 and the RAM 25 of the patient terminal 20. Accordingly, the description is omitted.

The operation receiver 32, which includes a keyboard including cursor keys, character input keys and various function keys, and a pointing device such as a mouse, outputs, to the controller 31, an operation signal input through key operation on the keyboard and mouse operation.

In a case where the healthcare worker terminal 30 is a tablet terminal or the like, the operation receiver 32 includes an operation key, such as a power key that turns on and off the power source, and a touch panel stacked on the display 33, and outputs, to the controller 31, an operation signal corresponding to each operation key and an operation signal in accordance with the position of a touch operation of a finger or the like of an operator.

The storage 36 includes an HDD or a nonvolatile semiconductor memory, and stores various processing programs (or instructions), and parameters, files and the like required to execute the programs. For example, the storage 36 stores a web browser program for achieving a web browser, a healthcare worker application and the like.

The storage 36 stores the electronic prescription information.

The electronic prescription information is information on a prescription issued to a patient (medicines, timing, quantities, etc.).

The electronic prescription information may be input by the healthcare worker through the healthcare worker terminal 30, or automatically captured from an electronic health record in the medical facility.

The storage 36 stores integrated information and statistical information received from the patient information management server 10.

### [Configuration of Pharmacy Terminal]

FIG. 5 shows the functional configuration of the pharmacy terminal 40.

As illustrated in FIG. 5, the pharmacy terminal 40 includes a controller 41, an operation receiver 42, a display 43, a communicator 44, a RAM 45 and a storage 46, which are connected to each other by a bus.

The controller 41, the operation receiver 42, the display 43, the communicator 44 and the RAM 45 of the pharmacy terminal 40 are respectively similar to the controller 31, the operation receiver 32, the display 33, the communicator 34 and the RAM 35 of the healthcare worker terminal 30. Accordingly, the description is omitted.

The storage 46 includes an HDD or a nonvolatile semiconductor memory, and stores various processing programs (or instructions), and parameters, files and the like required to execute the programs. For example, the storage 46 stores a web browser program for achieving a web browser, a pharmacy application and the like.

The storage 46 stores the dispensing information.

### [Configuration of Electronic Prescription Server]

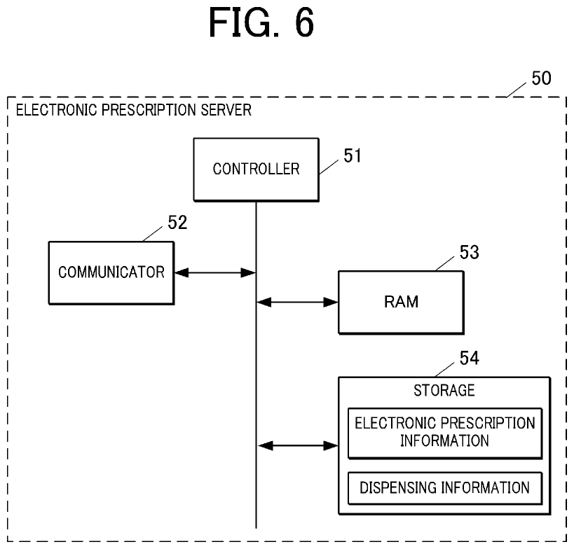

FIG. 6 shows the functional configuration of the electronic prescription server 50.

As shown in FIG. 6, the electronic prescription server 50 includes a controller 51, a communicator 52, a RAM 53 and a storage 54, which are connected to each other by a bus.

The controller 51 includes a CPU, and comprehensively controls processing operations of the components of the electronic prescription server 50. Specifically, the CPU reads various processing programs (or instructions) stored in the storage 54, loads the processing programs into the RAM 53 and executes various processes in cooperation with the programs.

The communicator 52 and the RAM 53 of the electronic prescription server 50 are similar to the communicator 12 and the RAM 13 of the patient information management server 10. Accordingly, the description is omitted.

The storage 54 includes an HDD or a nonvolatile semiconductor memory, and stores various processing programs (or instructions), and parameters, files and the like required to execute the programs.

The storage 54 stores the electronic prescription information registered from the healthcare worker terminal 30, and the dispensing information that is associated with the electronic prescription information and has been registered from the pharmacy terminal 40, with respect to each patient ID.

### [Operations of Information Processing System]

Next, information processing executed by the patient information management server 10 in the information processing system 100 is described.

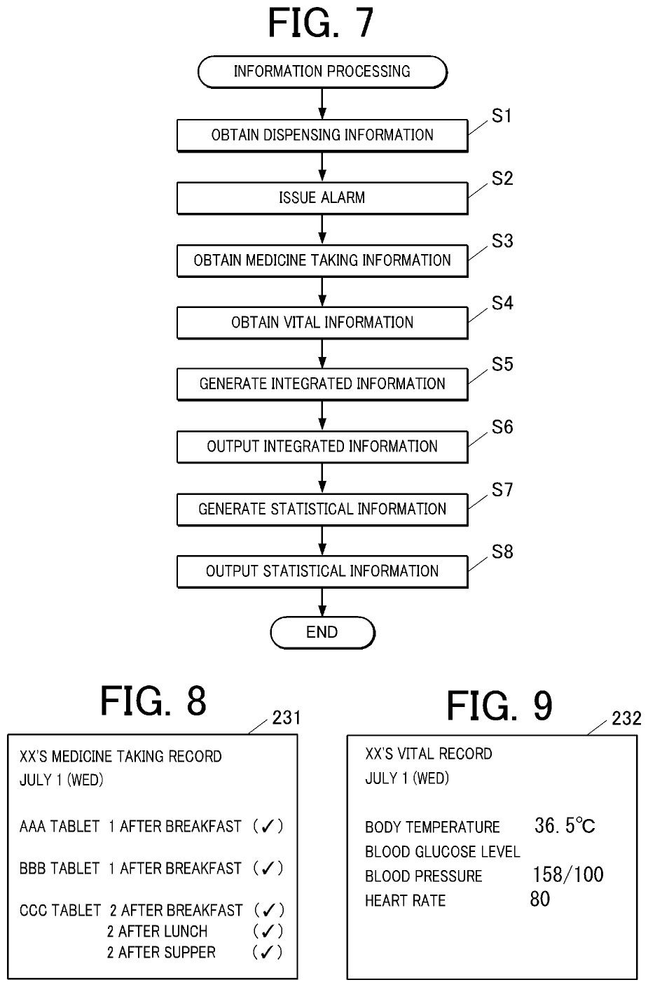

FIG. 7 shows a flowchart showing the flow of information processing.

The controller 11 of the patient information management server 10 obtains, from the patient terminal 20, dispensing information that is associated with the electronic prescription information and has been registered from one or more pharmacy terminals 40 (step S1). This step S1 is a first obtaining step.

The controller 11 stores, in the storage 14, the dispensing information obtained from the patient terminal 20 in association with the patient information on the patient associated with the patient terminal 20.

Next, the controller 11 delivers, as an alarm, medicine taking timing of each medicine to the patient application of the patient terminal 20, at medicine taking timing of the corresponding medicine (after breakfast, after lunch, after supper and the like), based on the dispensing information obtained in step S1 (step S2).

Next, the controller 11 causes the display 23 of the patient terminal 20 to display a medicine taking recording screen 231 (see FIG. 8) of the patient application at the medicine taking timing of each medicine, and accepts an input of the medicine taking information from the patient. That is, the controller 11 obtains medicine taking information on a patient from the patient terminal 20 (step S3). This step S3 is a second obtaining step.

The medicine taking recording screen 231 is created based on the dispensing information obtained in step S1.

FIG. 8 shows an example of the medicine taking recording screen 231.

As shown in FIG. 8, the quantity of medicine to be taken, and the medicine taking timing are displayed on the medicine taking recording screen 231 with respect to each medicine to be taken by the patient, thus allowing the patient to operate the operation receiver 22 and record presence or absence of medicine taking on the current day. The patient checks at the medicine taking timing (after breakfast, after lunch, after supper, etc.) of each medicine, thus inputting a fact that the medicine has already been taken.

The controller 21 of the patient terminal 20 transmits the medicine taking information input by the patient, to the patient information management server 10 via the communicator 24. The controller 11 of the patient information management server 10 stores the medicine taking information in the storage 14 in association with the patient information on the patient associated with the patient terminal 20.

Next, the controller 11 causes the display 23 of the patient terminal 20 to display a vital recording screen 232 of the patient application (see FIG. 9) and accepts an input of the vital information from the patient. That is, the controller 11 obtains the vital information from the patient terminal 20 (step S4). This step S4 is a third obtaining step.

FIG. 9 shows an example of the vital recording screen 232.

As shown in FIG. 9, the vital information on the patient is displayed on the vital recording screen 232. The vital information may be input from the patient via the operation receiver 22, or obtained from the vital measurement instrument via the Bluetooth communicator 27.

The controller 21 of the patient terminal 20 transmits the vital information input by the patient, to the patient information management server 10 via the communicator 24. The controller 11 of the patient information management server 10 stores the vital information in the storage 14 in association with the patient information on the patient associated with the patient terminal 20.

Next, the controller 11 associates the dispensing information obtained from the patient terminal 20 in step S1 with the medicine taking information obtained from the patient terminal 20 in step S3, and creates integrated information (step S5). This step S5 is an association step.

Note that the controller 11 may associate the dispensing information, the medicine taking information and the vitals obtained in step S4 with each other, and create integrated information.

The controller 11 stores, in the storage 14, the created integrated information in association with the patient information on the patient associated with the patient terminal 20.

Next, the controller 11 outputs the integrated information generated in step S5 to the healthcare worker application of the healthcare worker terminal 30 or the patient application of the patient terminal 20 (step S6). This step S6 is a first output step.

Note that the controller 11 outputs, with the integrated information, the test information on the patient registered from the healthcare worker terminal 30 and stored in the storage 14, when outputting the integrated information.

The controller 31 of the healthcare worker terminal 30 receives, via the communicator 34, the integrated information output from the patient information management server 10, and stores the information in the storage 36.

The controller 21 of the patient terminal 20 receives, via the communicator 24, the integrated information output from the patient information management server 10, and stores the information in the storage 26.

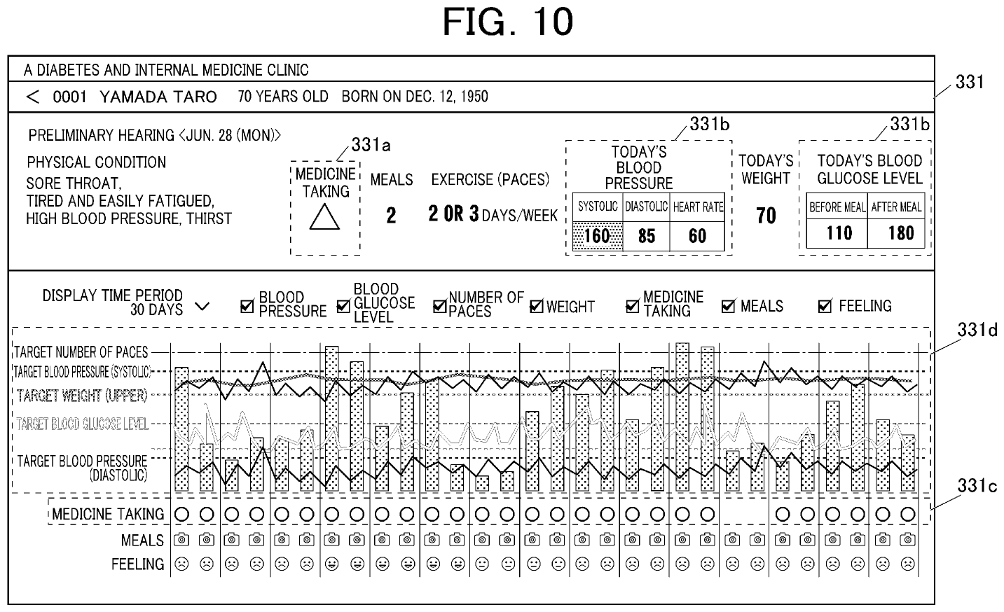

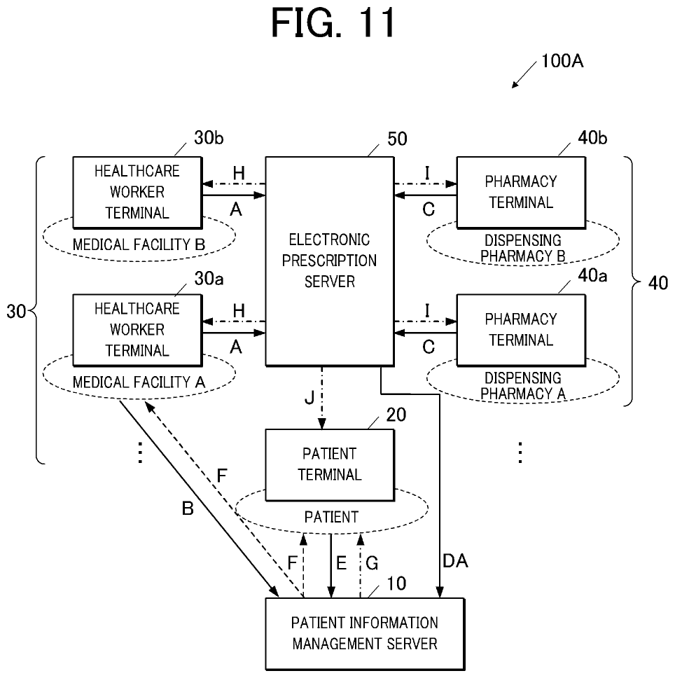

FIG. 10 shows an example of an integrated information screen 331 displayed on the display 33 of the healthcare worker terminal 30 for displaying the integrated information.

In the example shown in FIG. 10, the dispensing information is omitted.

The medicine taking information at a predetermined date (June 28 in the example shown in FIG. 10) is displayed on a medicine taking display 331a. The medicine taking information in a predetermined time period (30 days in the example shown in FIG. 10) is displayed on a medicine taking information display 331c in a time-series manner.

The vital information on the predetermined date (June 28 in the example shown in FIG. 10) is displayed on a vital display 331b. The vital information in the predetermined time period (30 days in the example shown in FIG. 10) is displayed on a vital information display 331d in a time-series manner.

Note that as shown in FIG. 10, the patient information (weight, age, etc.), exercise information, dietary information, physical condition information and the like on the patient may be displayed on the integrated information screen 331 with the integrated information. The exercise information, the dietary information and the physical condition information on the patient are information input by the patient through the patient terminal 20. The input information is transmitted to the patient information management server 10. The controller 11 of the patient information management server 10 then stores the input information in the storage 14 in association with the patient information on the patient associated with the patient terminal 20.

The dispensing information displayed on the integrated information screen 331 may include multiple pieces of dispensing information on prescriptions based on the electronic prescription information issued by multiple doctors.

The test information on the patient output with the integrated information, and a comment input by the doctor may be displayed on the integrated information screen 331.

An emergency screen that allows logon through input of an emergency code in case of an emergency may be created besides the integrated information screen 331. The dispensing information and the patient information (allergic information, side effect history, name of an injury or a disease, etc.) may be displayed on the emergency screen.

Next, the controller 11 applies statistical processing to the integrated information on multiple patients, and creates statistical information. That is, the controller 11 generates the statistical information, based on the integrated information (step S7). This step S7 is a generation step.

Next, the controller 11 outputs the statistical information generated in step S7 to the healthcare worker application of the healthcare worker terminal 30 or the patient application of the patient terminal 20 (step S8), and finishes the processing. This step S8 is a second output step.

The controller 31 of the healthcare worker terminal 30 receives, via the communicator 34, the statistical information output from the patient information management server 10, and stores the information in the storage 36.

The controller 21 of the patient terminal 20 receives, via the communicator 24, the statistical information output from the patient information management server 10, and stores the information in the storage 26.

Note that the controller 11 may execute step S7 before step S6, and output the statistical information with the integrated information in step S6.

Next, the aforementioned information processing in a case where the information processing system 100 includes a plurality of healthcare worker terminals 30 and a plurality of pharmacy terminals 40 is described.

For example, as shown in FIG. 1, a case is illustrated where the information processing system 100 includes: a healthcare worker terminal 30a (first healthcare worker terminal) that is a terminal used by a medical facility A (first medical facility); and a healthcare worker terminal 30b (second healthcare worker terminal) that is a terminal used by a medical facility B (second medical facility). Note that the unit of one facility is defined in units of insurance claims.

The information processing system 100 includes a pharmacy terminal 40a that is a terminal used by a dispensing pharmacy A, and a pharmacy terminal 40b that is a terminal used by a dispensing pharmacy B.

A doctor in the medical facility A records first electronic prescription information in the electronic prescription server 50 using the healthcare worker terminal 30a, as the operation A.

A doctor in the medical facility B records second electronic prescription information in the electronic prescription server 50 using the healthcare worker terminal 30b, as the operation A.

A pharmacist in the dispensing pharmacy A registers, in the electronic prescription server 50, first dispensing information associated with the first electronic prescription information registered from the healthcare worker terminal 30a, using the pharmacy terminal 40a, as the operation C.

A pharmacist in the dispensing pharmacy B registers, in the electronic prescription server 50, second dispensing information associated with the second electronic prescription information registered from the healthcare worker terminal 30b, using the pharmacy terminal 40b, as the operation C.

In this case, in step S1 in the information processing described above, the controller 11 obtains, from the patient terminal 20, (second) dispensing information associated with the second electronic prescription information registered (output) from the healthcare worker terminal 30b.

In step S6, the controller 11 outputs the integrated information to the healthcare worker application of the healthcare worker terminal 30a or the patient application of the patient terminal 20.

Consequently, the dispensing information that has been prescribed by another medical facility and dispensed by the pharmacy and conventionally has not been grasped can be obtained. Accordingly, any contraindicated combination of medicines to be provided for the patient can be avoided.

Alternatively, in step S1 in the information processing described above, the controller 11 may obtain, from the patient terminal 20, the (first) dispensing information associated with the first electronic prescription information registered (output) from the healthcare worker terminal 30a, and the (second) dispensing information associated with the second electronic prescription information registered (output) from the healthcare worker terminal 30b.

In this case, the electronic prescription server 50 transmits the first dispensing information and the second dispensing information to the patient terminal 20.

In the step S3 in the information processing described above, the controller 11 obtains, from the patient terminal 20, first medicine taking information associated with the first dispensing information. The controller 11 obtains second medicine taking information associated with the second dispensing information, from the patient terminal 20.

In the step S5 in the information processing described above, the controller 11 associates the first dispensing information with the first medicine taking information, and creates first integrated information. The controller 11 associates the second dispensing information with the second medicine taking information, and creates second integrated information.

In step S6 in the information processing described above, the controller 11 outputs the generated first integrated information and second integrated information, to the healthcare worker application of the healthcare worker terminal 30a, the healthcare worker application of the healthcare worker terminal 30b, or the patient application of the patient terminal 20.

As described above, according to the first embodiment, the patient information management server 10 is an information processing apparatus (patient information management server 10) that processes information obtained from the patient terminal 20 connectable to the electronic prescription server 50 that stores the electronic prescription information and the dispensing information and to the healthcare worker terminal 30, and includes: a first obtainer (controller 11) that obtains the dispensing information associated with the electronic prescription information from the patient terminal 20; a second obtainer (controller 11) that obtains the medicine taking information on the patient from the patient terminal 20; an association provider (controller 11) that associates the obtained dispensing information and medicine taking information with each other and creates the integrated information; and a first outputter (controller 11) that outputs the integrated information to the healthcare worker terminal 30 or the patient terminal 20.

In this way, one or more embodiments provide a practical improvement for correctly managing the dispensing information and the medicine taking information.

According to the first embodiment, the patient information management server 10 includes a third obtainer (controller 11) that obtains the vital information from the patient terminal 20. The integrated information includes the vital information.

Consequently, the doctor can confirm the effect of the medicine, based on the correlation between the medicine taking information and the vital information.

According to the first embodiment, the patient information management server 10 includes: a generator (controller 11) that generates the statistical information, based on the integrated information; and a second outputter (controller 11) that outputs the statistical information.

Consequently, the doctor can preferably confirm the effect of the medicine, based on the integrated information and the statistical information.

According to the first embodiment, the healthcare worker terminal 30 includes the first healthcare worker terminal (healthcare worker terminal 30a) and the second healthcare worker terminal (healthcare worker terminal 30b). The first obtainer obtains, from the patient terminal 20, the dispensing information associated with the electronic prescription information output from the second healthcare worker terminal, as the dispensing information associated with the electronic prescription information. The first outputter outputs the integrated information to the first healthcare worker terminal.

Consequently, the dispensing information that has been prescribed by another medical facility and dispensed by the pharmacy and conventionally has not been grasped can be obtained. Accordingly, any contraindicated combination of medicines to be provided for the patient can be avoided.

According to the first embodiment, in the patient information management server 10, the first obtainer obtains, from the patient terminal 20, the dispensing information associated with the electronic prescription information output from the first healthcare worker terminal, and the dispensing information associated with the electronic prescription information output from the second healthcare worker terminal, as the dispensing information associated with the electronic prescription information. The first outputter outputs the integrated information to the first healthcare worker terminal.

Consequently, the dispensing information associated with the electronic prescription information on prescriptions issued by the multiple medical facilities can be obtained.

According to the first embodiment, the first healthcare worker terminal is a terminal used by a first medical facility (medical facility A), and the second healthcare worker terminal is a terminal used by a second medical facility (medical facility B).

### Second Embodiment

Next, an information processing system 100A according to a second embodiment is described. Hereinafter, the points different from those in the information processing system 100 in the first embodiment are mainly described.

FIG. 11 shows the system configuration of an information processing system 100A.

In this embodiment, the patient information management server 10 is connected to the electronic prescription server 50, the patient terminal 20 and the healthcare worker terminal 30 via the communication network such as the Internet.

The controller 11 (hardware processor) of the patient information management server 10 in this embodiment obtains the dispensing information associated with the electronic prescription information, from the electronic prescription server 50. Here, the controller 11 functions as a first obtainer.

Next, operations of the information processing system 100A shown in FIG. 11 are described.

Instead of the operation D in FIG. 1, the electronic prescription server 50 transmits the dispensing information on the patient to the patient information management server 10 (operation DA).

As operation E, the patient registers data, such as the vital information and the medicine taking information, in the patient information management server 10, through the patient application using the patient terminal 20.

As the operation F, the patient information management server 10 transmits (outputs) integrated information that associates the dispensing information received from the electronic prescription server 50 with the medicine taking information, and statistical information generated based on the integrated information on multiple patients, to the patient terminal 20 and the healthcare worker terminal 30.

In step S1 in the information processing in this embodiment, the controller 11 obtains, from the electronic prescription server 50, the dispensing information that is associated with the electronic prescription information and has been registered from one or more pharmacy terminals 40.

Hereinafter, the controller 11 executes processes similar to those in the first embodiment described above.

Next, a case where the information processing system 100A includes a plurality of healthcare worker terminals 30 and a plurality of pharmacy terminals 40 is described.

For example, as shown in FIG. 11, a case is illustrated where the information processing system 100A includes: a healthcare worker terminal 30a (first healthcare worker terminal) that is a terminal used by a medical facility A (first medical facility); and a healthcare worker terminal 30b (second healthcare worker terminal) that is a terminal used by a medical facility B (second medical facility). Note that the unit of one facility is defined in units of insurance claims.

The information processing system 100A includes a pharmacy terminal 40a that is a terminal used by a dispensing pharmacy A, and a pharmacy terminal 40b that is a terminal used by a dispensing pharmacy B.

In this case, in step S1 in the information processing described above, the controller 11 obtains, from the electronic prescription server 50, (second) dispensing information associated with the second electronic prescription information registered (output) from the healthcare worker terminal 30b.

In step S6, the controller 11 outputs the integrated information to the healthcare worker application of the healthcare worker terminal 30a or the patient application of the patient terminal 20.

Alternatively, in step S1 in the information processing described above, the controller 11 may obtain, from the electronic prescription server 50, the (first) dispensing information associated with the first electronic prescription information registered (output) from the healthcare worker terminal 30a, and the (second) dispensing information associated with the second electronic prescription information registered (output) from the healthcare worker terminal 30b.

In the step S3 in the information processing described above, the controller 11 obtains, from the patient terminal 20, first medicine taking information associated with the first dispensing information. The controller 11 obtains second medicine taking information associated with the second dispensing information, from the patient terminal 20.

In the step S5 in the information processing described above, the controller 11 associates the first dispensing information with the first medicine taking information, and creates first integrated information. The controller 11 associates the second dispensing information with the second medicine taking information, and creates second integrated information.

In step S6 in the information processing described above, the controller 11 outputs the generated first integrated information and second integrated information, to the healthcare worker application of the healthcare worker terminal 30a, the healthcare worker application of the healthcare worker terminal 30b, or the patient application of the patient terminal 20.

As described above, according to the second embodiment, the patient information management server 10 is an information processing apparatus (patient information management server 10) that is connected to the electronic prescription server 50 that stores the electronic prescription information and the dispensing information, to the patient terminal 20 and to the healthcare worker terminal 30 and processes obtained information, and includes: a first obtainer (controller 11) that obtains the dispensing information associated with the electronic prescription information from the electronic prescription server 50; a second obtainer (controller 11) that obtains the medicine taking information on the patient from the patient terminal 20; an association provider (controller 11) that associates the obtained dispensing information and medicine taking information with each other and creates the integrated information; and a first outputter (controller 11) that outputs the integrated information to the healthcare worker terminal 30 or the patient terminal 20.

In this way, one or more embodiments provide a practical improvement for correctly managing the dispensing information and the medicine taking information.

According to the second embodiment, the healthcare worker terminal 30 includes the first healthcare worker terminal (healthcare worker terminal 30a) and the second healthcare worker terminal (healthcare worker terminal 30b). The first obtainer obtains, from the electronic prescription server 50, the dispensing information associated with the electronic prescription information output from the second healthcare worker terminal, as the dispensing information associated with the electronic prescription information. The first outputter outputs the integrated information to the first healthcare worker terminal.

Consequently, the dispensing information that has been prescribed by another medical facility and dispensed by the pharmacy and conventionally has not been grasped can be obtained. Accordingly, any contraindicated combination of medicines to be provided for the patient can be avoided.

According to the second embodiment, in the patient information management server 10, the first obtainer obtains, from the electronic prescription server 50, the dispensing information associated with the electronic prescription information output from the first healthcare worker terminal, and the dispensing information associated with the electronic prescription information output from the second healthcare worker terminal, as the dispensing information associated with the electronic prescription information. The first outputter outputs the integrated information to the first healthcare worker terminal.

Consequently, the dispensing information associated with the electronic prescription information on prescriptions issued by the multiple medical facilities can be obtained.

### Third Embodiment

Next, an information processing system 100B (healthcare worker system) according to a third embodiment is described. Hereinafter, the points different from those in the information processing system 100 in the first embodiment are mainly described.

In the information processing system 100B in this embodiment, the healthcare worker terminal 30 functions as an information processing apparatus.

The controller 31 (hardware processor) of the healthcare worker terminal 30 obtains the dispensing information associated with the electronic prescription information, from the electronic prescription server 50. Here, the controller 31 functions as a first obtainer.

The controller 31 obtains medicine taking information on a patient from the patient terminal 20. Here, the controller 11 functions as a second obtainer.

The controller 31 associates the obtained dispensing information and medicine taking information with each other and creates integrated information. Here, the controller 31 functions as an association provider.

The controller 31 outputs the integrated information to the display 33 of the healthcare worker terminal 30 or the patient terminal 20. Here, the controller 31 functions as a first outputter.

The controller 31 obtains vital information from the patient terminal 20. Here, the controller 31 functions as a third obtainer.

The controller 31 generates statistical information, based on the integrated information. Here, the controller 31 functions as a generator.

The controller 31 outputs the statistical information. Here, the controller 31 functions as a second outputter.

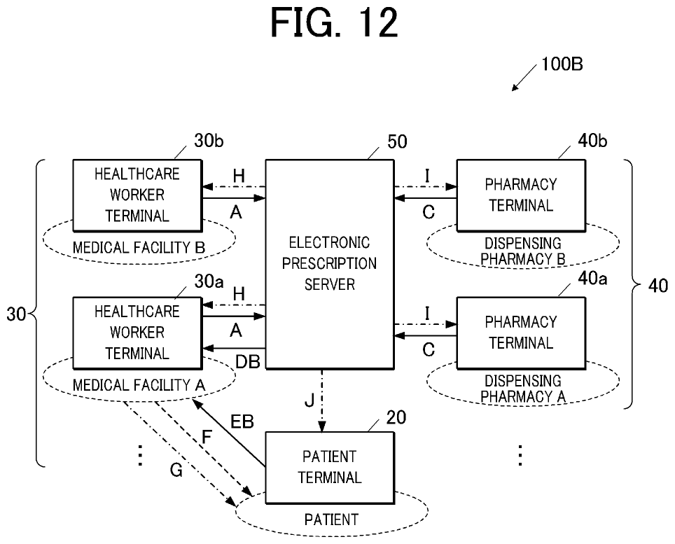

FIG. 12 shows the system configuration of the information processing system 100B.

Operations of the information processing system 100B shown in FIG. 12 are described.

Instead of the operation B in FIG. 1, the doctor (healthcare worker) stores data, such as test information, in association with the patient information, in the storage 36, using the healthcare worker terminal 30.

Instead of the operation D in FIG. 1, the electronic prescription server 50 transmits the dispensing information on the patient to the healthcare worker terminal 30 (operation DB).

Instead of the operation E in FIG. 1, the patient registers data, such as the vital information and the medicine taking information, in the healthcare worker terminal 30 through the patient application, using the patient terminal 20 (operation EB).

As the operation F, the healthcare worker terminal 30 transmits (outputs) integrated information that associates the dispensing information received from the electronic prescription server 50 and the medicine taking information received from the patient terminal 20 with each other, and statistical information generated based on the integrated information on multiple patients, to the patient terminal 20 and the healthcare worker terminal 30.

In the operation F, the healthcare worker terminal 30 displays the integrated information on the display 33 of the healthcare worker terminal 30. Alternatively, the healthcare worker terminal 30 may display the integrated information on the display 33 of another healthcare worker terminal 30.

As the operation G, the healthcare worker terminal 30 outputs an alarm for the patient to the patient terminal 20 via the patient application.

The healthcare worker terminal 30 executes information processing shown in FIG. 7 in the information processing system 100B.

The flow of the information processing by the healthcare worker terminal 30 is similar to the flow of the information processing by the patient information management server 10 described above. Accordingly, the description is omitted.

Next, a case where the information processing system 100B includes a plurality of healthcare worker terminals 30 and a plurality of pharmacy terminals 40 is described.

For example, as shown in FIG. 12, a case is illustrated where the information processing system 100B includes: a healthcare worker terminal 30a (first healthcare worker terminal) that is a terminal used by a medical facility A (first medical facility); and a healthcare worker terminal 30b (second healthcare worker terminal) that is a terminal used by a medical facility B (second medical facility). Note that the unit of one facility is defined in units of insurance claims.

The information processing system 100B further includes a pharmacy terminal 40a that is a terminal used by a dispensing pharmacy A, and a pharmacy terminal 40b that is a terminal used by a dispensing pharmacy B.

In this case, in step S1 in the information processing described above, the controller 31 obtains, from the electronic prescription server 50, (second) dispensing information associated with the second electronic prescription information registered (output) from the healthcare worker terminal 30b.

In step S6, the controller 31 outputs the integrated information to the healthcare worker application of the healthcare worker terminal 30a or the patient application of the patient terminal 20.

Alternatively, in step S1 in the information processing described above, the controller 31 may obtain the (first) dispensing information associated with the first electronic prescription information registered (output) from the healthcare worker terminal 30a, and the (second) dispensing information associated with the second electronic prescription information registered (output) from the healthcare worker terminal 30b, from the electronic prescription server 50.

In the step S3 in the information processing described above, the controller 31 obtains, from the patient terminal 20, first medicine taking information associated with the first dispensing information. The controller 31 obtains, from the patient terminal 20, second medicine taking information associated with the second dispensing information.

In the step S5 in the information processing described above, the controller 31 associates the first dispensing information with the first medicine taking information, and creates first integrated information. The controller 31 associates the second dispensing information with the second medicine taking information, and creates second integrated information.

In step S6 in the information processing described above, the controller 31 outputs the generated first integrated information and second integrated information, to the healthcare worker application of the healthcare worker terminal 30a, the healthcare worker application of the healthcare worker terminal 30b, or the patient application of the patient terminal 20.

As described above, according to the third embodiment, the healthcare worker terminal 30 is the healthcare worker terminal 30 that processes information obtained from the electronic prescription server 50 that stores the electronic prescription information and the dispensing information and from the patient terminal 20, and includes: the first obtainer (controller 31) that obtains the dispensing information associated with the electronic prescription information from the electronic prescription server 50; the second obtainer (controller 31) that obtains the medicine taking information on the patient from the patient terminal 20; the association provider (controller 31) that associates the obtained dispensing information and medicine taking information with each other and creates the integrated information; and the first outputter (controller 31) that outputs the integrated information to the display 33 of the healthcare worker terminal 30 or the patient terminal 20.

In this way, one or more embodiments provide a practical improvement for correctly managing the dispensing information and the medicine taking information.

According to the third embodiment, the healthcare worker terminal 30 includes a third obtainer (controller 31) that obtains the vital information from the patient terminal 20. The integrated information includes the vital information.

Consequently, the doctor can confirm the effect of the medicine, based on the correlation between the medicine taking information and the vital information.

According to the third embodiment, the healthcare worker terminal 30 includes: a generator (controller 31) that generates the statistical information, based on the integrated information; and a second outputter (controller 31) that outputs the statistical information.

Consequently, the doctor can preferably confirm the effect of the medicine, based on the integrated information and the statistical information.

According to the third embodiment, the healthcare worker terminal 30 includes the first healthcare worker terminal (healthcare worker terminal 30a) and the second healthcare worker terminal (healthcare worker terminal 30b). The first obtainer obtains, from the electronic prescription server 50, the dispensing information associated with the electronic prescription information output from the second healthcare worker terminal, as the dispensing information associated with the electronic prescription information. The first outputter outputs the integrated information to the first healthcare worker terminal.

Consequently, the dispensing information that has been prescribed by another medical facility and dispensed by the pharmacy and conventionally has not been grasped can be obtained. Accordingly, any contraindicated combination of medicines to be provided for the patient can be avoided.

According to the third embodiment, in the healthcare worker terminal 30, the first obtainer obtains, from the electronic prescription server 50, the dispensing information associated with the electronic prescription information output from the first healthcare worker terminal, and the dispensing information associated with the electronic prescription information output from the second healthcare worker terminal, as the dispensing information associated with the electronic prescription information. The first outputter outputs the integrated information to the first healthcare worker terminal.

Consequently, the dispensing information associated with the electronic prescription information on prescriptions issued by the multiple medical facilities can be obtained.

Note that although the embodiments of the present invention have been described above, the present invention is not limited to such embodiments, and obviously various modifications are possible within a scope that does not depart from the gist of the present invention.

For example, the information processing systems 100, 100A and 100B in the embodiments described above assume that one healthcare worker terminal 30 is used in one medical facility. However, there is no limitation to such a configuration. A plurality of healthcare worker terminals 30 may be used in one medical facility.

In the first embodiment and the second embodiment, the patient information management server 10, which is the information processing apparatus, functions as the first obtainer, the second obtainer, third obtainer, the association provider, the first outputter, the second outputter and the generator. However, there is no limitation to such a configuration. The patient information management server 10 may function as any of the components described above, and the other apparatuses may function as the remaining components.

Further alternatively, detailed configurations and detailed operations of the components that constitute the information processing apparatus, the healthcare worker terminal, the information processing system and the healthcare worker system can be appropriately changed in a range without departing from the spirit of the present invention.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A non-transitory computer readable recording medium storing instructions to control a computer of an information processing apparatus that processes information obtained from a patient terminal connectable to a healthcare worker terminal and to an electronic prescription server that stores electronic prescription information and dispensing information, the instructions causing the computer to execute:
obtaining, from the patient terminal, the dispensing information associated with the electronic prescription information;
obtaining, from the patient terminal, medicine taking information on a patient;
associating the dispensing information and the medicine taking information with each other and creating integrated information; and
outputting the integrated information to the healthcare worker terminal or the patient terminal.

2. The recording medium according to claim 1, wherein
the instructions cause the computer to execute:
obtaining vital information from the patient terminal, and
the integrated information includes the vital information.

3. The recording medium according to claim 1, wherein
the instructions cause the computer to execute:
generating statistical information based on the integrated information; and
outputting the statistical information.

4. The recording medium according to claim 1, wherein
the healthcare worker terminal includes a first healthcare worker terminal and a second healthcare worker terminal, and
the instructions cause the computer to execute:
obtaining, from the second healthcare worker terminal via the patient terminal, the dispensing information associated with the electronic prescription information; and
outputting the integrated information to the first healthcare worker terminal.

5. The recording medium according to claim 4, wherein
the instructions cause the computer to execute:
obtaining, from the first healthcare worker terminal, the dispensing information associated with the electronic prescription information;
obtaining, from the second healthcare worker terminal via the patient terminal, the dispensing information associated with the electronic prescription information; and
outputting the integrated information to the first healthcare worker terminal.

6. The recording medium according to claim 4, wherein
the first healthcare worker terminal is a terminal used by a first medical facility, and
the second healthcare worker terminal is a terminal used by a second medical facility.

7. An information processing apparatus that processes information obtained from a patient terminal connectable to a healthcare worker terminal and to an electronic prescription server that stores electronic prescription information and dispensing information, the information processing apparatus comprising:
a hardware processor that:
obtains, from the patient terminal, the dispensing information associated with the electronic prescription information,
obtains, from the patient terminal, medicine taking information on a patient,
associates the dispensing information and the medicine taking information with each other and creates integrated information, and
outputs the integrated information to the healthcare worker terminal or the patient terminal.

8. The information processing apparatus according to claim 7, wherein
the hardware processor:
obtains vital information from the patient terminal, and
the integrated information includes the vital information.

9. The information processing apparatus according to claim 7, wherein
the hardware processor:
generates statistical information based on the integrated information, and
outputs the statistical information.

10. The information processing apparatus according to claim 7, wherein
the healthcare worker terminal includes a first healthcare worker terminal, and a second healthcare worker terminal, and
the hardware processor:
obtains, from the second healthcare worker terminal via the patient terminal, the dispensing information associated with the electronic prescription information, and
outputs the integrated information to the first healthcare worker terminal.

11. The information processing apparatus according to claim 10, wherein
the hardware processor:
obtains, from the first healthcare worker terminal, the dispensing information associated with the electronic prescription information,
obtains, from the second healthcare worker terminal via the patient terminal, the dispensing information associated with the electronic prescription information, and
outputs the integrated information to the first healthcare worker terminal.

12. The information processing apparatus according to claim 10, wherein
the first healthcare worker terminal is a terminal used by a first medical facility, and
the second healthcare worker terminal is a terminal used by a second medical facility.

13. A non-transitory computer readable recording medium storing instructions to control a computer of an information processing apparatus that processes obtained information and is connected to a patient terminal, to a healthcare worker terminal, and to an electronic prescription server that stores electronic prescription information and dispensing information, the instructions causing the computer to execute:
obtaining, from the electronic prescription server, the dispensing information associated with the electronic prescription information;
obtaining, from the patient terminal, medicine taking information on a patient;
associating the dispensing information and the medicine taking information with each other and creating integrated information; and
outputting the integrated information to the healthcare worker terminal or the patient terminal.

14. The recording medium according to claim 13, wherein
the instructions cause the computer to execute:
obtaining vital information from the patient terminal, and
the integrated information includes the vital information.

15. The recording medium according to claim 13, wherein
the instructions cause the computer to execute:
generating statistical information based on the integrated information; and
outputting the statistical information.

16. The recording medium according to claim 13, wherein
the healthcare worker terminal includes a first healthcare worker terminal and a second healthcare worker terminal, and
the instructions cause the computer to execute:
obtaining, from the second healthcare worker terminal via the electronic prescription server, the dispensing information associated with the electronic prescription information; and
outputting the integrated information to the first healthcare worker terminal.

17. The recording medium according to claim 16, wherein
the instructions cause the computer to execute:
obtaining, from the first healthcare worker terminal, the dispensing information associated with the electronic prescription information;
obtaining, from the second healthcare worker terminal via the electronic prescription server, the dispensing information associated with the electronic prescription information; and
outputting the integrated information to the first healthcare worker terminal.

18. The recording medium according to claim 16, wherein
the first healthcare worker terminal is a terminal used by a first medical facility, and
the second healthcare worker terminal is a terminal used by a second medical facility.

19. An information processing apparatus that processes obtained information and is connected to a patient terminal, to a healthcare worker terminal, and to an electronic prescription server that stores electronic prescription information and dispensing information, the information processing apparatus comprising:
a hardware processor that:
obtains, from the electronic prescription server, the dispensing information associated with the electronic prescription information,
obtains, from the patient terminal, medicine taking information on a patient,
associates the dispensing information and the medicine taking information with each other and creates integrated information, and
outputs the integrated information to the healthcare worker terminal or the patient terminal.

20. The information processing apparatus according to claim 19, wherein
the hardware processor:
obtains vital information from the patient terminal, and
the integrated information includes the vital information.

21. The information processing apparatus according to claim 19, wherein
the hardware processor:
generates statistical information based on the integrated information, and
outputs the statistical information.

22. The information processing apparatus according to claim 19, wherein
the healthcare worker terminal includes a first healthcare worker terminal and a second healthcare worker terminal, and
the hardware processor:
obtains, from the second healthcare worker terminal via the electronic prescription server, the dispensing information associated with the electronic prescription information, and
outputs the integrated information to the first healthcare worker terminal.

23. The information processing apparatus according to claim 22, wherein
the hardware processor:
obtains, from the first healthcare worker terminal, the dispensing information associated with the electronic prescription information,
obtains, from the second healthcare worker terminal via the electronic prescription server, the dispensing information associated with the electronic prescription information, and
outputs the integrated information to the first healthcare worker terminal.

24. The information processing apparatus according to claim 22, wherein
the first healthcare worker terminal is a terminal used by a first medical facility, and
the second healthcare worker terminal is a terminal used by a second medical facility.

25. A non-transitory computer readable recording medium storing instructions to control a computer of a healthcare worker terminal that processes information obtained from a patient terminal and from an electronic prescription server that stores electronic prescription information and dispensing information, the instructions causing the computer to execute:
obtaining, from the electronic prescription server, the dispensing information associated with the electronic prescription information;
obtaining, from the patient terminal, medicine taking information on a patient;
associating the dispensing information and the medicine taking information with each other and creating integrated information; and
outputting the integrated information to a display of the healthcare worker terminal or the patient terminal.

26. The recording medium according to claim 25, wherein
the instructions cause the computer to execute:
obtaining vital information from the patient terminal, and
the integrated information includes the vital information.

27. The recording medium according to claim 25, wherein
the instructions cause the computer to execute:
generating statistical information based on the integrated information; and
outputting the statistical information.

28. The recording medium according to claim 25, wherein
the healthcare worker terminal includes a first healthcare worker terminal, and a second healthcare worker terminal, and
the instructions cause the computer to execute:
obtaining, from the second healthcare worker terminal via the electronic prescription server, the dispensing information associated with the electronic prescription information; and
outputting the integrated information to the first healthcare worker terminal.

29. The recording medium according to claim 28, wherein
the instructions cause the computer to execute:
obtaining, from the first healthcare worker terminal, the dispensing information associated with the electronic prescription information;
obtaining, from the second healthcare worker terminal via the electronic prescription server, the dispensing information associated with the electronic prescription information; and
outputting the integrated information to the first healthcare worker terminal.

30. The recording medium according to claim 28, wherein
the first healthcare worker terminal is a terminal used by a first medical facility, and
the second healthcare worker terminal is a terminal used by a second medical facility.

31. A healthcare worker terminal that processes information obtained from a patient terminal and from an electronic prescription server that stores electronic prescription information and dispensing information, the healthcare worker terminal comprising:
a hardware processor that:
obtains, from the electronic prescription server, the dispensing information associated with the electronic prescription information,
obtains, from the patient terminal, medicine taking information on a patient,
associates the dispensing information and the medicine taking information with each other and creates integrated information, and
outputs the integrated information to a display of the healthcare worker terminal or the patient terminal.

32. The healthcare worker terminal according to claim 31, wherein
the hardware processor:
obtains vital information from the patient terminal, and
the integrated information includes the vital information.

33. The healthcare worker terminal according to claim 31, wherein
the hardware processor:
generates statistical information based on the integrated information, and
outputs the statistical information.

34. The healthcare worker terminal according to claim 31, wherein
the healthcare worker terminal includes a first healthcare worker terminal, and a second healthcare worker terminal, and
the hardware processor:
obtains, from the second healthcare worker terminal via the electronic prescription server, the dispensing information associated with the electronic prescription information, and
outputs the integrated information to the first healthcare worker terminal.

35. The healthcare worker terminal according to claim 34, wherein
the hardware processor:
obtains, from the first healthcare worker terminal, the dispensing information associated with the electronic prescription information,
obtains, from the second healthcare worker terminal via the electronic prescription server, the dispensing information associated with the electronic prescription information, and
outputs the integrated information to the first healthcare worker terminal.

36. The healthcare worker terminal according to claim 34, wherein
the first healthcare worker terminal is a terminal used by a first medical facility, and
the second healthcare worker terminal is a terminal used by a second medical facility.

37. An information processing system comprising:
an information processing apparatus that processes information obtained from a patient terminal connectable to a healthcare worker terminal and to an electronic prescription server that stores electronic prescription information and dispensing information; and
a hardware processor that:
obtains, from the patient terminal, the dispensing information associated with the electronic prescription information,
obtains, from the patient terminal, medicine taking information on a patient,
associates the dispensing information and the medicine taking information with each other and creates integrated information, and
outputs the integrated information to the healthcare worker terminal or the patient terminal.

38. An information processing system comprising:
an information processing apparatus that processes obtained information and is connected to a patient terminal, to a healthcare worker terminal, and to an electronic prescription server that stores electronic prescription information and dispensing information; and
a hardware processor that:
obtains, from the electronic prescription server, the dispensing information associated with the electronic prescription information,
obtains, from the patient terminal, medicine taking information on a patient,
associates the dispensing information and the medicine taking information with each other and creates integrated information, and
outputs the integrated information to the healthcare worker terminal or the patient terminal.

39. A healthcare worker system comprising:
a healthcare worker terminal that processes information obtained from a patient terminal and from an electronic prescription server that stores electronic prescription information and dispensing information; and
a hardware processor that:
obtains, from the electronic prescription server, the dispensing information associated with the electronic prescription information,
obtains, from the patient terminal, medicine taking information on a patient,
associates the dispensing information and the medicine taking information with each other and creates integrated information, and
outputs the integrated information to the healthcare worker terminal or the patient terminal.
